(19) United States
(12) Patent Application Publication
(10) Pub. No.: US 2019/0242846 A1
(43) Pub. Date: Aug. 8, 2019

(54) DEVICES AND METHODS FOR CREATION AND CALIBRATION OF A NANOELECTRODE PAIR

(71) Applicants: Quantum Biosystems Inc., Tokyo (JP); Osaka University, Osaka (JP)

(72) Inventors: Masateru TANIGUCHI, Osaka (JP); Takahito Ohshiro, Osaka (JP); Makusu Tsutsui, Osaka (JP); Kazumichi Yokota, Osaka (JP); Toshiki Matsuoka, Tokyo (JP); Mark Oldham, Emerald Hills, CA (US); Eric Nordman, Palo Alto, CA (US)

(21) Appl. No.: 16/266,363

(22) Filed: Feb. 4, 2019

Related U.S. Application Data
(63) Continuation of application No. PCT/JP2017/027962, filed on Aug. 2, 2017.
(60) Provisional application No. 62/370,175, filed on Aug. 2, 2016.

(51) Int. Cl. G01N 27/327; G01N 33/487

(52) U.S. Cl. CPC G01N 27/3278; B82Y 40/00; G01N 33/48721

(57) ABSTRACT

Methods and systems are provided for creation of stable and consistent nanoelectrode pairs for detection of biomolecules, such as deoxyribonucleic acid.

### CROSS-REFERENCE

This application claims the benefit of U.S. Provisional Patent Application Ser. No. 62/370,175, filed Aug. 2, 2016, which is entirely incorporated herein by reference.

### BACKGROUND ART

Nanopores may be useful for determining the sequence of a nucleic acid molecule, such as a deoxyribonucleic acid (DNA) or ribonucleic acid (RNA) molecule. The determination of the sequence of a nucleic acid molecule may provide various benefits, such as aiding in diagnosing and/or treating a subject. For example, the nucleic acid sequence of a subject may be used to identify, diagnose and potentially develop treatments for genetic diseases.

Nanoelectrode pairs used for measuring the current across molecules and tunneling current have been described as being useful for determining the sequence of biopolymers, such as single stranded DNA.

### SUMMARY OF INVENTION

The present disclosure provides methods and apparatuses for creating nanoelectrode pairs suitable for various applications, such as biopolymer detection and sequencing, including nucleic acid sequencing, sequencing other biopolymers, and detection and identification of other molecules. Nanoelectrode pairs of the present disclosure may be used for detection or sequencing of deoxyribonucleic acid (DNA) or ribonucleic acid (RNA), and variants thereof. Provided herein are improved methods to fabricate nanoelectrode pairs and calibrate a gap spacing of nanoelectrode pairs.

An aspect of the present disclosure provides a method of forming a nano-gap electrode pair, comprising: (a) providing a metal substrate having a region configured to form a gap; (b) applying a voltage across the region of the metal substrate; and (c) applying tensile stress on the region of the metal substrate to form the gap by controlling an expansion rate of the region along a direction of the tensile stress, wherein the tensile stress is applied until a conductance of the metal substrate is less than 7 G₀.

In some embodiments, (c) is performed while measuring the conductance of the metal substrate. In some embodiments, the expansion rate is controlled automatically. In some embodiments, the method further comprises controlling the expansion rate such that there is a decrease and an increase of the expansion rate. In some embodiments, the metal substrate is a metal wire. In some embodiments, (c) comprises: (i) when the conductance is greater than or equal to 7 G₀, applying the tensile stress to provide a first expansion rate of the region; (ii) when the conductance is less than 7 G₀, applying the tensile stress to provide a second expansion rate of the region, which second expansion rate is greater than the first expansion rate; and (iii) when the conductance is less than or equal to 3 G₀, applying the tensile stress to provide a third expansion rate of the region, which third expansion rate is less than the second expansion rate.

In some embodiments, the tensile stress is applied to provide the third expansion rate when the conductance is between 1 G₀ and 3 G₀. In some embodiments, when the conductance is less than or equal to 3 G₀, the tensile stress is applied such that the third expansion rate is substantially zero. In some embodiments, when the conductance is less than or equal to 3 G₀, the tensile stress is applied such that the third expansion rate is non-zero. In some embodiments, when the conductance approaches G₀, the tensile stress is applied to provide a fourth expansion rate of the region that is substantially zero. In some embodiments, the second expansion rate is provided for a time period of less than 10 seconds. In some embodiments, the second expansion rate is provided for a time period of less than 3 seconds. In some embodiments, the second expansion rate is provided for a time period of less than 2 seconds. In some embodiments, the metal substrate is formed by lithography. In some embodiments, the metal substrate is less than 100 nm wide at the region. In some embodiments, the gap has a spacing from 0.5 nanometers (nm) to 2 times a molecular diameter of a biomolecule. In some embodiments, the spacing is from 0.5 nm to a molecular diameter of the biomolecule. In some embodiments, the gap has a spacing from 0.5 nm to less than a molecular diameter of a biomolecule.

Another aspect of the present disclosure provides a method of setting a G₀ gap distance between a pair of nanoelectrodes of a mechanically controlled break junction, comprising: (a) applying a voltage between the pair of nanoelectrodes; (b) varying a gap distance between the pair of nanoelectrodes, which varying includes applying one or more cycles of alternating motion to increase and reduce the gap distance, and wherein individual electrodes of the pair of nanoelectrodes do not reconnect when the gap distance is reduced; (c) measuring a current between the pair of nanoelectrodes: and (d) calculating the gap distance as a function of a plurality of data sets of the gap distance and the current.

In some embodiments, the current includes tunneling current. In some embodiments, the varying comprises a deceleration of a rate at which the gap distance is varied prior to a reversal in a direction in which the gap distance is varied. In some embodiments, the plurality of data sets comprises data generated by motion in the same direction as an intended or predetermined motion.

Another aspect of the present disclosure provides a method for forming a nanoelectrode pair, comprising: (a) forming a metal wire coated with an electrically insulating material; and (b) forming the nanoelectrode pair from the metal wire, wherein the nanoelectrode pair has a gap with a spacing from about 0.5 nanometers (nm) to 10 nm.

In some embodiments, the electrically insulating material is thermally insulating. In some embodiments, molecules of the electrically insulating material are bonded to multiple atoms of the metal wire. In some embodiments, (b) comprises subjecting the metal wire to stress. In some embodiments, the metal wire is subjected to stress upon application of force to a region of the metal wire. In some embodiments, the nanoelectrode pair has a gap that is configured to detect a current upon flow of a biomolecule through the gap. In some embodiments, the gap has a spacing from 0.5 nanometers (nm) to 2 times a molecular diameter of the biomolecule.

Another aspect of the present disclosure provides a method for forming a gap spacing in a nanoelectrode pair, comprising: (a) providing the nanoelectrode pair having tips; (b) rejoining the tips of the nanoelectrode pair; (c) rebreaking the nanoelectrode pair to provide the tips; and (d) repeating (b) and (c) at least five times, thereby creating a reformed nanoelectrode pair having the gap spacing.

In some embodiments, (b) and (c) are repeated more than ten times. In some embodiments, (b) and (c) are repeated more than twenty times. In some embodiments, (a) comprises providing a metal wire and breaking the metal wire. In some embodiments, the breaking and rejoining are performed using an actuator. In some embodiments, the breaking and rejoining are performed using an actuator, and wherein an average velocity of the actuator during the breaking is less than an average velocity of the actuator during the rejoining.

Another aspect of the present disclosure provides a biomolecule analysis apparatus comprising a nanoelectrode pair, wherein the nanoelectrode pair was formed by breaking of a metal wire, wherein the breaking is done on an apparatus separate from the biomolecule analysis apparatus.

Another aspect of the present disclosure method for providing a reformed nanoelectrode pair, comprising: (a) providing a nanoelectrode pair having a degraded performance, which degraded performance is characterized by increased background noise, wherein the nanoelectrode pair includes separate electrodes with tips; (b) rejoining the tips of the nanoelectrode pair to form a rejoined unit; (c) breaking the rejoined unit to reform the nanoelectrode pair; and (d) repeating (b) and (c) at least once to provide the reformed nanoelectrode pair.

In some embodiments, the method further comprises, prior to (b) measuring the degraded performance in the nanoelectrode pair. In some embodiments, (d) comprises repeating (b) and (c) at least twice. In some embodiments, (d) comprises repeating (b) and (c) at three times. In some embodiments, the reformed nanoelectrode pair has a gap with a spacing from 0.5 nanometers (nm) to 2 times a molecular diameter of a biomolecule.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A illustrates a schematic representation of a nanoelectrode forming bridge prior to breakage.

FIG. 1B illustrates a schematic representation of a nanoelectrode forming bridge after breakage.

FIG. 1C illustrates a schematic representation of the atomic arrangement of a G₀ junction prior to breakage.

FIG. 2A shows a photomicrograph of an uncoated nanoelectrode forming bridge with a wide nanochannel without a top cover.

FIG. 2B shows a photomicrograph of a coated nanoelectrode forming bridge with a narrow nanochannel formed in the coating but without a top cover.

FIG. 3 shows a plot of tunneling current from an uncoated nanoelectrode forming bridge and a coated nanoelectrode forming bridge.

FIG. 4 shows a control panel from an automated system for forming nanoelectrode pairs.

FIG. 5 shows data generated from an automated system for forming nanoelectrode pairs.

FIG. 6A shows current data from a simple linear fit approach for calibration of the gap spacing of a nanoelectrode pair.

FIG. 6B shows a representation of the piezo voltage versus time for a conventional system.

FIG. 7A shows current data from an example method of the present disclosure for calibration of the gap spacing of a nanoelectrode pair.

FIG. 7B shows a representation of the piezo voltage versus time for an example method of the present disclosure for calibration of the gap spacing of a nanoelectrode pair.

FIG. 7C shows another representation of the piezo current versus time for an example method of the present disclosure for calibration of the gap spacing of a nanoelectrode pair.

FIG. 8A shows data from a manual setup using a conventional approach.

FIG. 8B shows data from an automated setup using an example method of the present disclosure.

FIG. 9 shows a table comparing performance metrics of an example method of the present disclosure versus a conventional approach.

FIG. 10 shows a distribution of gap spacing after breaking using an example method of the present disclosure versus a conventional method.

FIG. 11 shows a schematic illustration of a mechanically controllable break junction (MCBJ) apparatus.

FIG. 12A shows a schematic illustration of a calibration curve for a gap control actuator wherein the gap is not a linear function of the actuator control signal.

FIG. 12B shows another schematic illustration of a calibration curve for a gap control actuator, in which the gap is not a linear function of the actuator control signal.

FIG. 13 schematically illustrates a computer system that is programmed or otherwise configured to implement devices, systems and methods of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure is not limited to specific compositions, method steps, or equipment. It is also to be understood that the terminology used herein is for the purpose of describing particular embodiments only, and is not intended to be limiting. Methods recited herein may be carried out in any order of the recited events that is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

It must be noted that, as used in this specification and the appended claims, the singular forms “a”, “an” and “the” include plural forms, unless the context clearly dictates otherwise. Thus, for example, reference to “a biopolymer” includes more than one biopolymer. In describing and claiming the present invention, the following terminology will be used in accordance with the definitions set out below.

The term “gap,” as used herein, generally refers to a pore, channel or passage formed or otherwise provided in a material. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1,000 nm. A gap having a width on the order of nanometers may be referred to as a “nano-gap” (also “nanogap” herein). In some situations, a nano-gap has a width that is from about 0.1 nanometers (nm) to 50 nm, 0.5 nm to 30 nm, or 0.5 nm to 10 nm, 0.5 nm to 5 nm, or 0.5 nm to 2 nm, or no greater than about 2 nm, 1 nm, 0.9 nm, 0.8 nm, 0.7 nm, 0.6 nm, or 0.5 nm. In some cases, a nano-gap has a width that is at least about 0.5 nm, 0.6 nm, 0.7 nm, 0.8 nm, 0.9 nm, 1 nm, 2 nm, 3 nm, 4 nm, or 5 nm. In some cases, the width of a nano-gap can be less than a diameter of a biomolecule or a subunit (e.g., monomer) of the biomolecule.

The term “conductance quantum,” as used herein, is designated as G₀, and is a quantized unit of electrical conduction defined by the equation: G₀=2e²/h where ‘e’ is the elementary charge and ‘h’ is Planck's constant. A G₀ gap is a gap in which a conductance of G₀ has been effectuated during the formation of the gap, for example during a stretching process.

The term “electrode,” as used herein, generally refers to a material or part that can be used to measure electrical current. An electrode (or electrode part) can be used to measure electrical current to or from another electrode. In some situations, electrodes can be disposed in a channel (e.g., nanogap) and be used to measure the current across the channel. The current can be a tunneling current. Such a current can be detected upon the flow of a biomolecule (e.g., protein) through the nano-gap. In some cases, a sensing circuit coupled to electrodes provides an applied voltage across the electrodes to generate a current. As an alternative or in addition to, the electrodes can be used to measure and/or identify the electric conductance associated with a biomolecule (e.g., an amino acid subunit or monomer of a protein). In such a case, the tunneling current can be related to the electric conductance.

In some examples, a nanoelectrode pair includes individual nanoelectrodes that are separated by a gap with spacing less than or equal to about 50 nm, 40 nm, 30 nm, 20 nm, 10 nm, 5 nm, 4 nm, 3 nm, 2 nm, 1 nm, or 0.5 nm. A nanoelectrode may have any convenient shape or size and can be comprised of any conductive material. Each electrode of the present disclosure can be fabricated from different materials or from mixtures of materials such as alloys.

Nanoelectrodes may be used for the measurement of a current which may travel through and/or across a molecule. The current may be a tunneling current. Measurements of current may be used for determining the sequence of a biopolymer, such as a nucleic acid molecule (e.g., DNA or RNA) or a protein. For quality measurements, the gap spacing between the electrodes of one or more nanoelectrode pairs may be stable and controllable.

The term “nanoelectrode forming bridge,” as used herein, generally refers to a conductive connection which may be broken to create a nanoelectrode pair. The conductive connection may be a lithographically patterned metal, a localized nanowire(s), a conductive polymer, which may be a linear polymer, such as polyacetylene, polypyrrole or polyaniline, in some cases when the linear polymer is doped or protonated.

The term “nanochannel,” as used herein, general refers to a covered trench with at least one pair of nanoelectrodes intersecting the nanochannel. A channel may have any convenient shape or size, in some cases with varying width and depth.

The term “biomolecule,” as used herein generally refers to any biological material that can be interrogated with an electrical current and/or potential across a nano-gap electrode. A biomolecule can be a nucleic acid molecule, protein, or carbohydrate. A biomolecule can include one or more subunits, such as nucleotides or amino acids. In some examples, a biomolecule is a biopolymer, which is any polymer in an organism and may include deoxyribonucleic acid (DNA), ribonucleic acid (RNA), a protein, a lipid chain and a polysaccharide. A biopolymer may be a polymer which may be a modified version of a polymer from an organism. Biopolymers such as DNA, RNA, a protein, lipid or polysaccharide may be sequenced. Biopolymers other than DNA may be sequenced. In the present disclosure, any mention of DNA can be replaced by another biopolymer, and vice versa.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U. or any other subunit that is specific to one or more complementary A, C, G, T or U, or complementary to a purine (i.e., A or G, or variant thereof) or a pyrimidine (i.e., C. T or U. or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded.

The term “protein,” as used herein, generally refers to a biological molecule, or macromolecule, having one or more amino acid monomers, subunits or residues. A protein containing 50 or fewer amino acids, for example, may be referred to as a “peptide.” The amino acid monomers can be selected from any naturally occurring and/or synthesized amino acid monomer, such as, for example, 20, 21, or 22 naturally occurring amino acids. In some cases, 20 amino acids are encoded in the genetic code of a subject. Some proteins may include amino acids selected from about 500 naturally and non-naturally occurring amino acids. In some situations, a protein can include one or more amino acids selected from isoleucine, leucine, lysine, methionine, phenylalanine, threonine, tryptophan and valine, arginine, histidine, alanine, asparagine, aspartic acid, cysteine, glutamine, glutamic acid, glycine, proline, serin and tyrosine.

The term “substrate,” as used herein, refers to any workpiece on which film or thin film formation is desired. A substrate includes, without limitation, silicon, germanium, silica, sapphire, zinc oxide, carbon (e.g., graphene), SiC, AlN, GaN, spinel, coated silicon, silicon on oxide, silicon carbide on oxide, glass, gallium nitride, indium nitride, titanium dioxide and aluminum nitride, a ceramic material (e.g., alumina, AlN), a metallic material (e.g., molybdenum, tungsten, copper, aluminum), and combinations (or alloys) thereof. A substrate can include a single layer or multiple layers. A substrate may be conducting or non-conducting.

A mechanically controllable break junction (MCBJ) system may provide a way to generate nanoelectrode tips with monoatomic points and adjustable gap spacing. Existing methods may provide a low yield, as not all methods result in a usable monoatomic junction. In addition, nanoelectrode pair performance may be substantially susceptible to small changes. The background tunneling current across a nanoelectrode pair gap may change at least about 10 fold with only an Angstrom change in the gap spacing between electrodes of a nanoelectrode pair. Background noise (e.g., background current) may increase relative to a baseline. The baseline may be a predetermined level at which the performance of the nanoelectrode pair has been found to be preferable or optimum, such as preferable or optimum signal to noise. Metal atoms may move if the local atomic structure is not stable. Migration of ions may significantly change the gap spacing and consequently the instrument performance. A nanoelectrode gap may need to be set to a desired or predetermined spacing.

It may be desirable to create nanoelectrode pairs, such as MCBJ nanoelectrode pairs, or other adjustable nanoelectrode pairs with high yield, suitable gap spacing stability and tightly controlled gap spacing. For best or optimal performance, background noise associated with ionic current may be minimized.

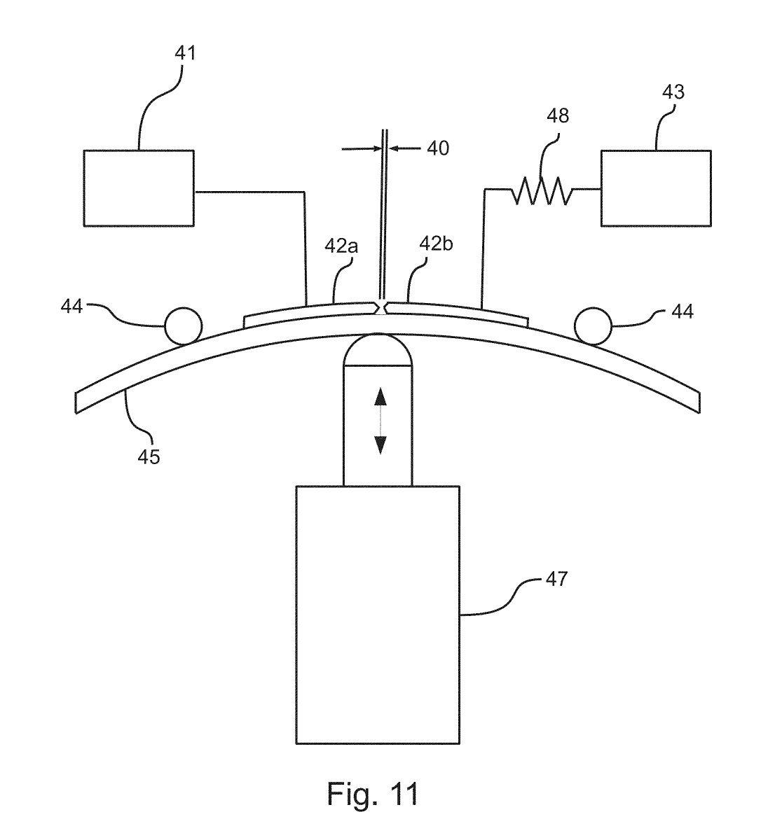

FIG. 11 shows a schematic illustration of a mechanism that may be used to create a stable nanoelectrode pair with a given, desired or predetermined tip gap spacing. A linear actuator 47 may be used to deform substrate 45 by bending substrate 45 against two supports 44.

A linear actuator may be operated in an open loop mode. As an alternative, a linear actuator may have positional feedback, allowing it to operate in a closed loop mode.

A substrate 45 may be electrically nonconducting or may have an electrically insulating coating. Deflection of substrate 45 may cause nanoelectrode forming bridge (not shown) to break, forming nanoelectrodes 42a and 42b and nanoelectrode gap spacing 40. If linear actuator 47 is retracted, nanoelectrodes 42a and 42b may be rejoined. This may yield a rejoined unit. The rejoined unit may be a single unit. The linear actuator 47 may be applied to the nanoelectrodes 42a and 42b at a velocity. The velocity may be controlled. If linear actuator 47 is extended nanoelectrodes 42a and 42b may be rebroken. Because the stretching of the top surface of substrate 45 is small compared to the motion of linear actuator 47, a displacement demagnification may occur, allowing sub Angstrom positional control of the nanoelectrode gap spacing.

In FIG. 11 a bias voltage source 41 may be connected to nanoelectrode 42a. Current flowing between nanoelectrodes 42a and 42b may be measured by a low current ammeter 43 such as a trans-impedance amplifier which may be directly associated with a voltmeter or any other appropriate current or charge measuring device. In some cases, a voltmeter may be configured to be separate from the low current ammeter 43. Between nanoelectrode 42b and low current ammeter 43, a protective resistor (Rp) 48 may be used. In some cases, the protective resistor may be positioned between bias voltage source 41 and nanoelectrode 42a, or between both bias voltage source 41 and nanoelectrode 42a and nanoelectrode 42b and low current ammeter 43. Protective resistor 48 may limit the current when nanoelectrodes 42a and 42b are joined and may have a value of 1 kΩ (kilo ohms) to 10 kΩ, or may have a value of 10 kΩ to 100 kΩ, or may have a value of 100 kΩ to 1 MΩ, or may have a value of 1 MΩ to 10 MΩ or may have a value of greater than 1 kΩ, 10 kΩ, 100 kΩ, 1 MΩ, or 10 MΩ. An output from low current ammeter 43 may be recorded by suitable electronics (not shown).

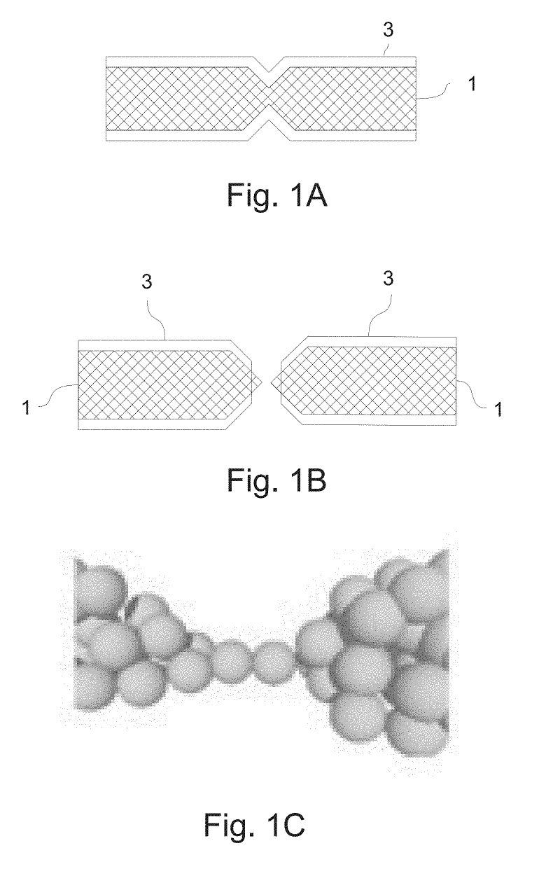

In some cases the background current and associated noise may be reduced by minimizing the surface area of the nanoelectrode in fluidic contact. FIG. 1A shows a schematic of a nanoelectrode forming bridge 1, and an insulating coating 3. FIG. 1B shows the nanoelectrode forming bridge 3 after the bridge has been broken by stretched exposing metal of a nanoelectrode pair. In some cases the nanoelectrode forming bridge may be broken by a mechanically controlled break junction apparatus. In some cases the actuation motion of the mechanically controlled break junction apparatus can be provided by a piezo element, piezo driven actuator, inchworm piezo actuators, motor driven mechanism or combinations thereof. FIG. 1C schematically illustrates a possible atomic arrangement of the neck of the stretched nanoelectrode forming bridge 1 just prior to breaking.

In some cases the nanoelectrode forming bridge may be coated, e.g., by an electrically insulating material. In some cases the insulating coating may be both electrically insulating and thermally insulating. In some cases the nanoelectrode tips may be exposed by breaking of the nanoelectrode forming bridge. In some cases, lithography may be used to expose a section of the metal. In some cases the insulating coating may substantially reduce the amount of exposed metal, but may expose more metal than just the electrode atomic tips. In some cases the insulating coating material may be insulators commonly used in semiconductor processing such as SiO2, Si3N4, resists or polymeric materials such as plastics. In some cases the coating may be a conformal coating. In some cases, the coating may be hydrophilic to facilitate wetting of the nanochannel, thereby directing an aqueous solution containing one or more biopolymers towards the nanoelectrode.

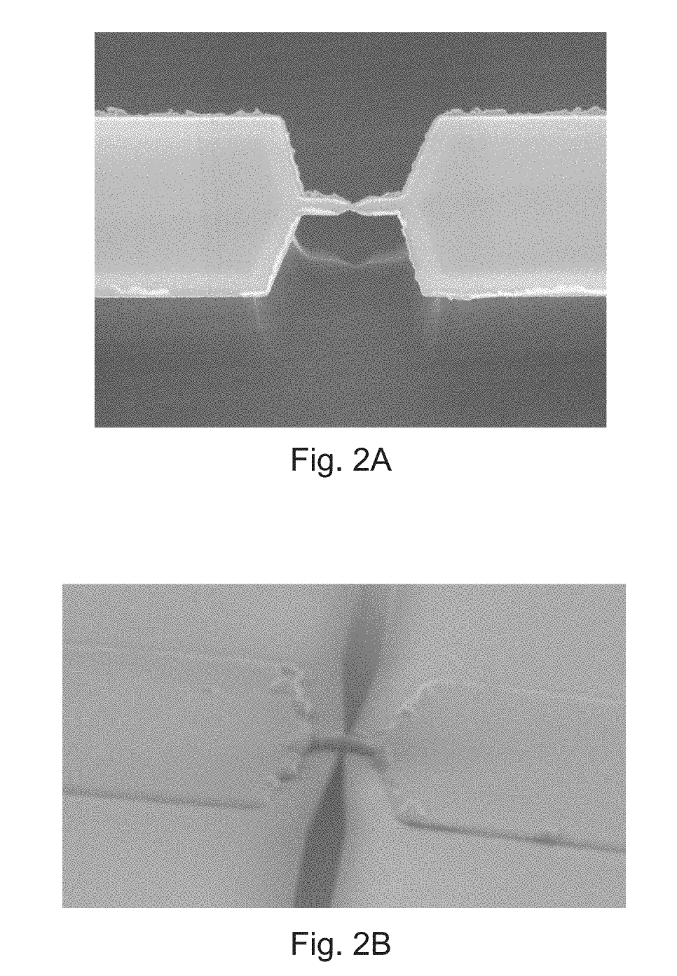

FIG. 2A shows a photomicrograph of an uncoated nanoelectrode forming bridge with a wide nanochannel without a top cover. FIG. 2B shows a photomicrograph of a coated nanoelectrode forming bridge with a narrow nanochannel formed in the coating and the insulating material below, but without a top cover.

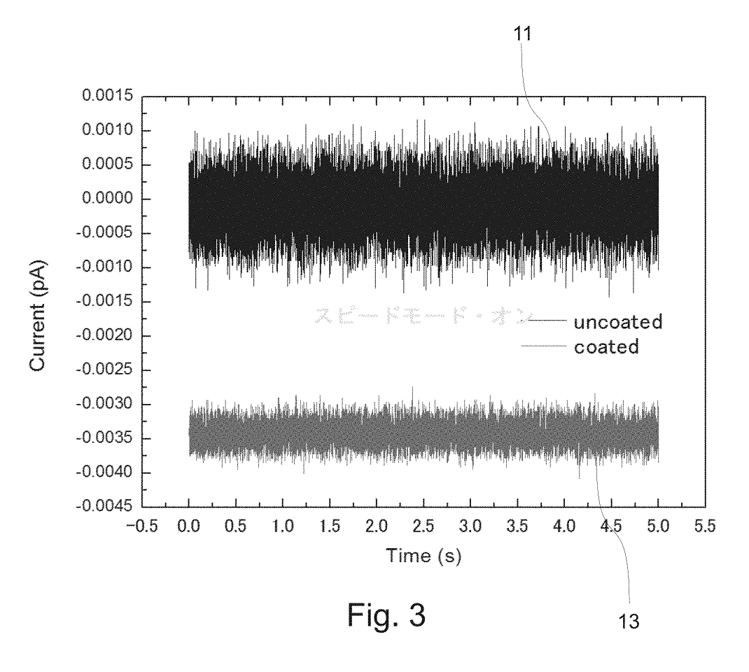

FIG. 3 shows a plot of the background current for an uncoated channel 11, and the background current for a coated channel 13. As clearly seen in the plot, the noise of the background is higher in the current from uncoated channel 11 as compared to the coated channel 13. The average background is higher for the uncoated channel, but this is masked by offsets in the plotted current measurement. All of the currents would be positive without the offset.

The gap spacing between the tips of a nanoelectrode pair may be consistent over time and set to the desired or predetermined gap spacing. The gap spacing may be significantly influenced by motions of atoms. Less stable atoms can move under the influence of the electron wind, generated by the current flowing across the nanoelectrode pair. Because of this, the crystalline structure of the tip may be important. The structure of the tip may depend on how the tip is created. Nanoelectrode pairs may be fabricated by stretching a nanoelectrode forming bridge. In some cases a mechanically controllable break junction (MCBJ) setup may be used to create nanoelectrodes. In some cases, the nanoelectrode pairs may be reconnected and broken multiple times (e.g., greater than or equal to about 2, 3, 4, 5, 6, 7, 8, 9, 10, 11, 12, 13, 14, 15, 16, 17, 18, 19, 20, 25, 30, 35, 40, 45, 50, or more times) in order to create a local crystalline structure at the tip. In some cases, the resulting crystalline structure at the tip may have a (111) crystalline configuration. In some cases, the ability to consistently regenerate G₀ nanoelectrode pairs, after repeated joining and rebreaking (e.g., automated reciprocating motion), may be used to determine when a desired or predetermined crystalline tip structure has been created. In some cases, the automated reciprocating motion may be repeated a predetermined number of times. A predetermined number of times may have be an experimentally determined value that properly weighs tip stability and tip creation time. In some cases the predetermined number of times may be a fixed number greater than or equal to about 5, 10, 20, 50, 100, 200, 500, 1,000 or more. In some cases, one or more criteria may be utilized to determine a number of times for utilizing an automated reciprocating motion, wherein in a criterion may be a current level, a change (such as an increase or a decrease) in current level, or other appropriate criteria.

Different methods of generating nanoelectrodes using MCBJ systems have been described. See, for example, Tsutsui et al., “Fabrication of 0.5 nm electrode gaps using self-breaking technique,” Applied Physics Letters 93.16 (2008): 163115 (“Tsutsui”), and U.S. Pat. No. 5,751,156 to Muller et al., each of which is entirely incorporated herein by reference. While these methods can generate nanoelectrode pairs, they may be slow and the yield may be low.

The present disclosure provides various approaches for improving the fabrication and calibration of nanoelectrode pairs. In some cases this may be done by changing the stretching rate. In some cases the nanoelectrode forming bridge is stretched until the conductance drops to less than or equal to 15 G₀, 14 G₀, 13 G₀, 12 G₀, 11 G₀, 10 G₀, 9 G₀, 8 G₀, 7 G₀, 6 G₀, 5 G₀ or less. At this point the stretching (or expansion) rate may be increased. In some cases, as the nanoelectrode forming bridge gets closer to breaking, the stretching rate may be decelerated. In some embodiments, this deceleration may occur when the conductance drops to less than or equal to 8 G₀, 7 G₀, 6 G₀, 5 G₀, 4 G₀, 3 G₀, 2 G₀ or less. In some cases, the conductance associated with this deceleration is less than the conductance associated with the increase in stretching rate. The increasing and decreasing of stretching rates may be automated.

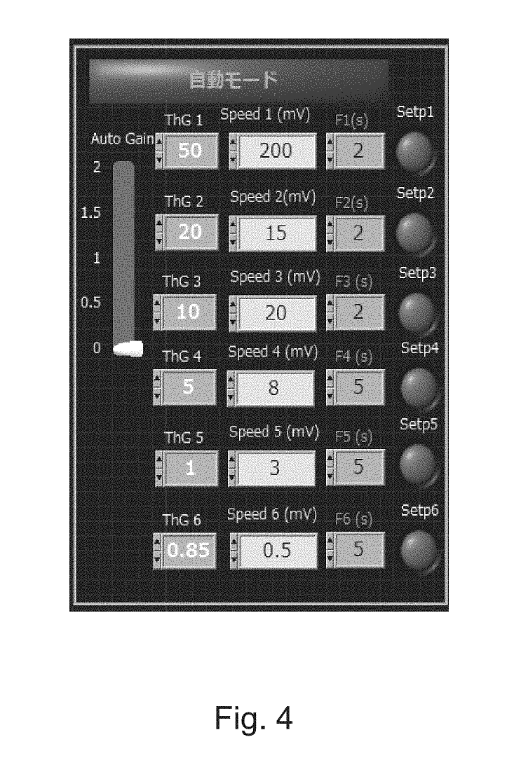

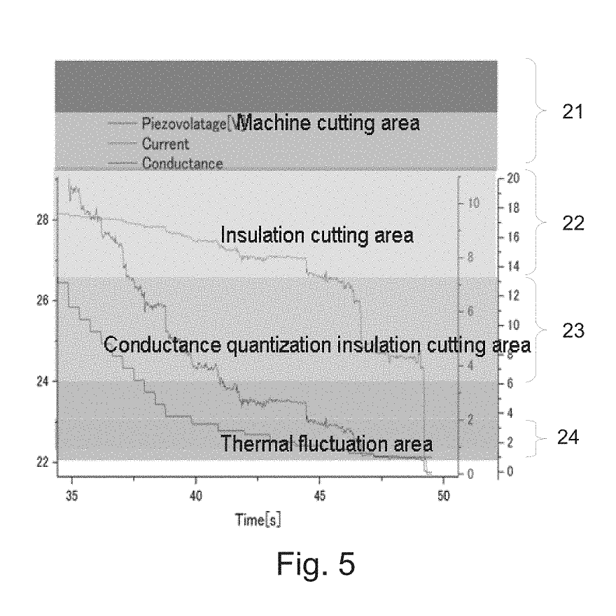

FIG. 4 shows an example control panel used to control a piezo actuator. The piezo actuator may be used to control the stretching, breaking and joining of one or more MCBJs, allowing control of distance and time during multiple steps. The control panel may include a graphical user interface (GUI). Transitions from one step to another may be based on measurement of the conductance. FIG. 5 shows a set of conductances curves which illustrate some step, as described herein. In the machine cutting step 21 the stretching steps are large as controlled by the first set point in FIG. 4 which may be 200/2 millivolts (mV)/second (sec) or 100 mV/sec. The expansion steps are in mV as these are changes to the piezo actuator which converts voltage changes in to small displacement changes. When the conductance drops to 50 G₀ the software may transition to the insulation cutting area 22 and the speed or expansion rate may be slowed to 15/2 mV/sec or 7.5 mV/sec, and may proceed at this rate until the conductance drops to 20 G₀. The speed may then be increased to 20/2 mV/sec or 10 mV/sec until the conductance drops to 10 G₀. It may then further slow to 8/5 mV/sec or 1.6 mV/sec until the conductance drops to 5 G₀. It may then further slow to 3/5 mV/sec or 0.6 mV/sec until the conductance drops to 1 G₀ as part of the thermal fluctuation area 24. At this time, it may slow considerably to 0.5/5 mV/sec or 0.1 mV/sec until the conductance drops to 0.85 G₀. The atomic connection then breaks in part due to thermal oscillation fluctuations.

In some cases, the maximum value can be up to 20 G₀. For rejoining and breaking (tip training), when the tips get rejoined, the conductance may be about 20 G₀. After rejoining, the gap distance may be increased and the stretching (or expansion or separation) rate may be increased. The conductance of 20 G₀ may be lower than the conductance after the rejoining subsequent to any breaking.

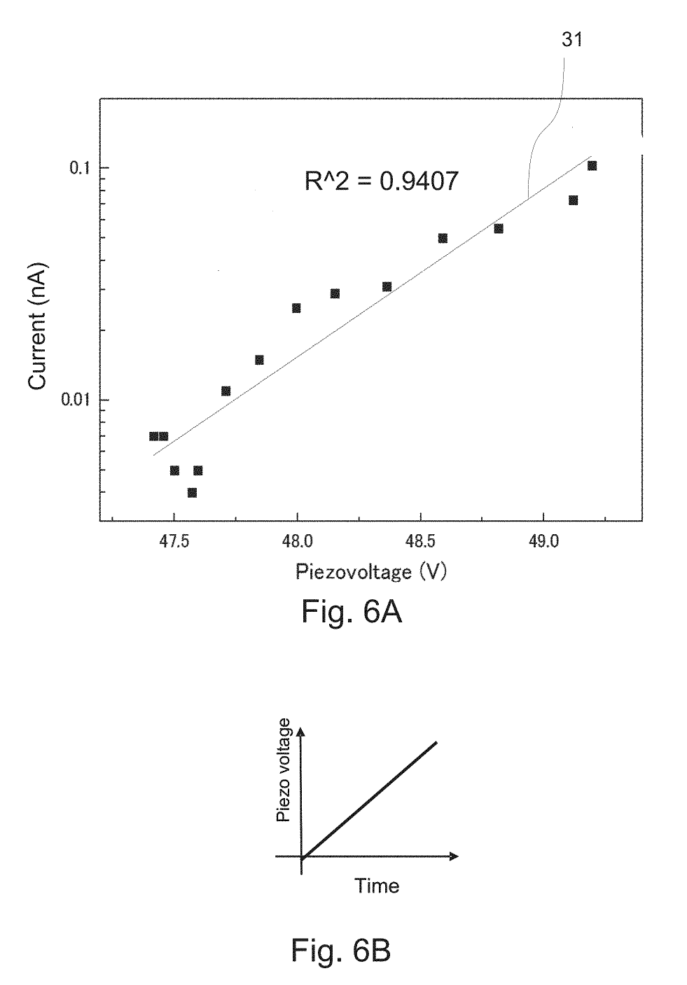

The rate of nanoelectrode gap spacing change as a function of actuator motion may be accurately calibrated. FIG. 6B shows a schematic representation of a simple linear fit calibration approach. Previously the measurement of tunneling current was done by sweeping the voltage in a single direction after breakage. FIG. 6A shows a calibration linear fit 31 to the log-linear data which has an R2 value of 0.9047. For tunneling measurements, the amount of tunneling current is expected to be proportional to exp(B*d) where B is a constant, and d is the gap spacing between the single atoms comprising the tips of the nanoelectrodes. In an MCBJ system driven by a conventional piezo actuator, the vertical displacement may be proportional to the voltage (or negative voltage depending on actuator design) and gap displacement may be proportional to vertical displacement. Consequently, if tunneling current is plotted on a log scale versus piezo voltage, a straight line is expected. The calibration slope of this function may be used to calibrate the gap spacing. For a standard piezo mechanism, a hysteresis may be typically observed, wherein upon changing from a positive dV/dt to a negative dV/dt, a period without observable motion may occur despite potentially significant changes in the voltage applied. For a piezo motor or Inchworm™ type of piezo mechanism, the calibration may depend on the number of steps the Piezo motor or Inchworm™ is driven, wherein the number of steps per vertical displacement may be different depending on the direction the Piezo or Inchworm™ is driven.

While a piezo actuator is used for this data, any linear actuator may be used to drive the vertical displacement of an MCBJ system. While an MCBJ system may be mounted so the actuator is driven vertically, it is obvious that any orientation may be used.

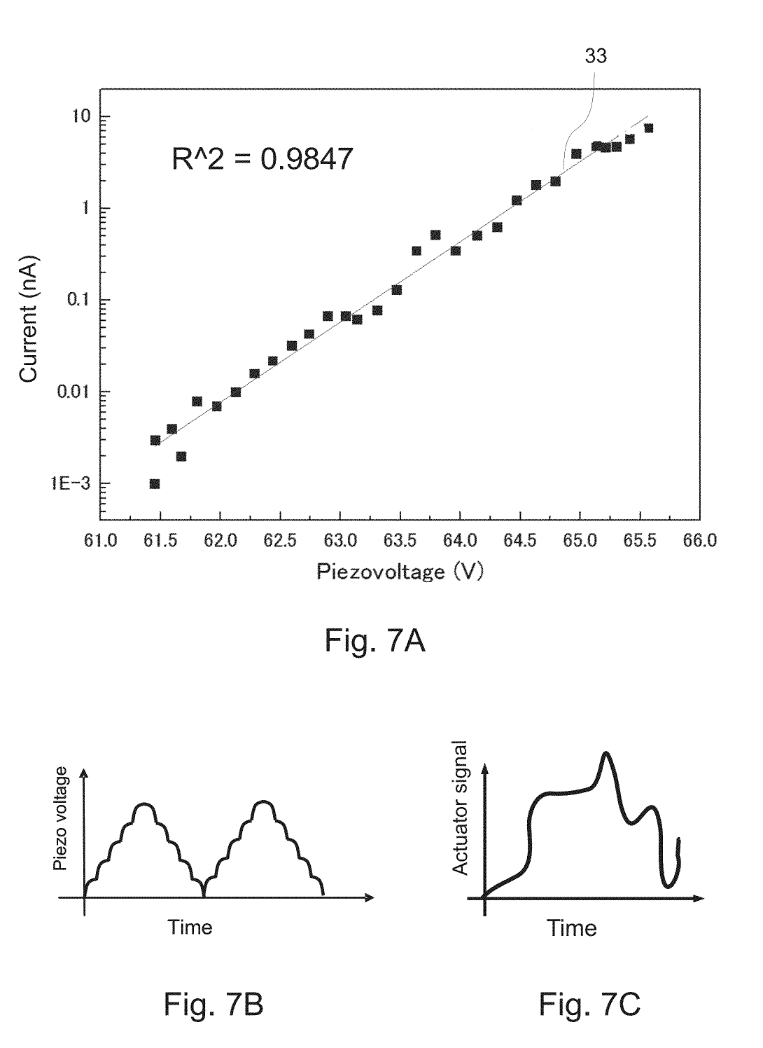

In some cases, slope calibration may be improved upon by collecting data during one or more cycles of changing of the gap spacing, wherein the gap spacing may be both increased and decreased one or more times without rejoining of the electrodes of the nanoelectrode pair. This profile is illustrated in 7B where the gap control is performed using a voltage driven piezo device. In some cases, as shown in FIG. 7C the reversals of direction profile can be any function.

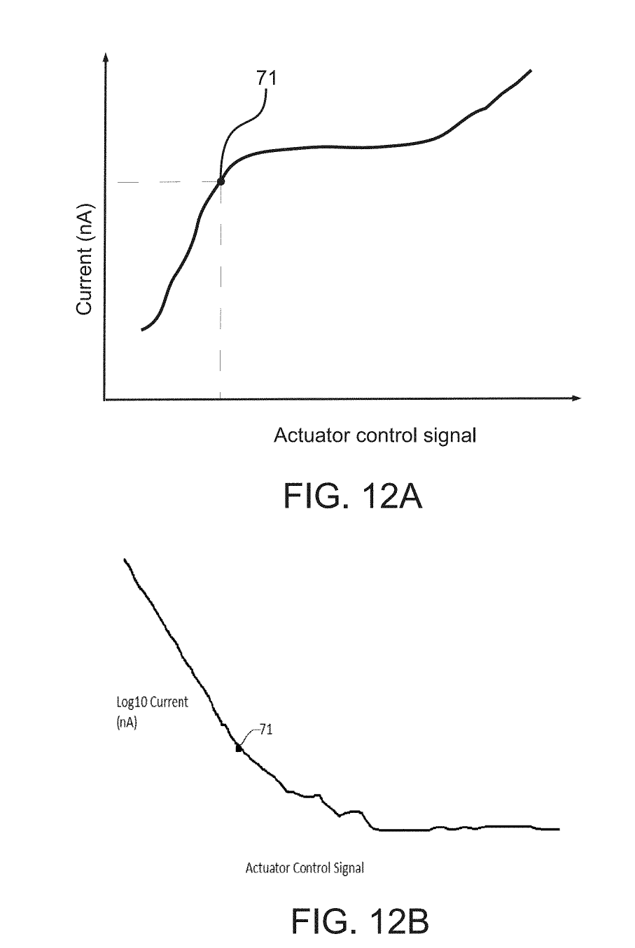

In some cases, the gap spacing may not be a linear function of the actuator signal. When gap control is not a linear function of the actuator signal, a plot of actuator signal versus current, log(current), or other function of current may be generated. Such a plot, as illustrated in FIG. 12A, may not be linear, but may be used with a calibration curve derived from a setup where the gap distance to current relationship is known, or may be determined as a result of measurement of tunneling current. This may allow the determination of the relationship between a nonlinear actuator signal and gap displacement. In some cases, a position 71 where a displacement is known may be used to determine a position offset. Position 71 may be determined using tunneling currents without a sample being present as described elsewhere herein.

FIG. 12B shows another example of a plot of current as a function of actuator control signal. A lower actuator signal may correspond with a higher current. With increasing actuator signal, the current may asymptotically approach a constant value.

In some cases, the switching of directions is done slowly. A sine wave, radiused triangular profile, and trapezoidal profile are examples of sweep patterns wherein the switching of directions may be done slowly. In some cases a deceleration/acceleration time may occur in a time less than or equal to one half an alternating motion period.

In some cases, the slope may be calculated for both directions. This may be useful, as hysteresis may be significant in some systems. In some cases, a slope used for calibration may be utilized from data generated by motion in the same direction as the intended motion.

In some cases, data points near the reversal point may be excluded, in determining the calibration slope.

FIG. 7A shows a plot of data from multiple direction sweeps. The improved calibration fit 33 is a linear fit to the data. As can be seen the R2 fit is much improved over the value from FIG. 6A. In some cases, the quality of a linear fit may be used to determine whether a good tip structure has been generated.

In some cases the nanoelectrode gap spacing may be reduced until the tips are joined, at which time the nanogap may be adjusted to a desired or predetermined gap spacing using a slope calibration factor. In some cases, a nanoelectrode gap spacing setting may be set by starting with an expected break distance and using a slope calibration factor to determine the correct setting to achieve the desired or predetermined gap spacing.

In some cases, a time period from joining to breakage of the fracture surfaces, may be made longer than the time period from breakage to joining of the fracture surfaces. A reason that a breaking time may be set longer, may be to accurately detect a breaking point, when the current values, etc., may reach a threshold value which may be determined to provide optimal tip formation. A rejoining motion may be made faster, as measurement accuracy is not required. Accordingly, a complete processing time may be reduced, while accurately detecting breakage of the fine metal wire.

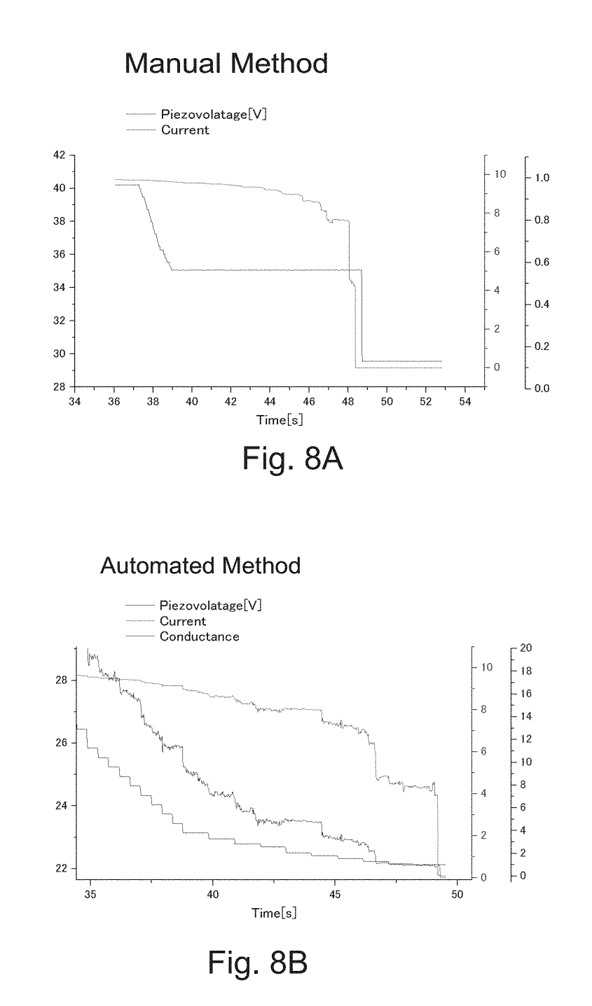

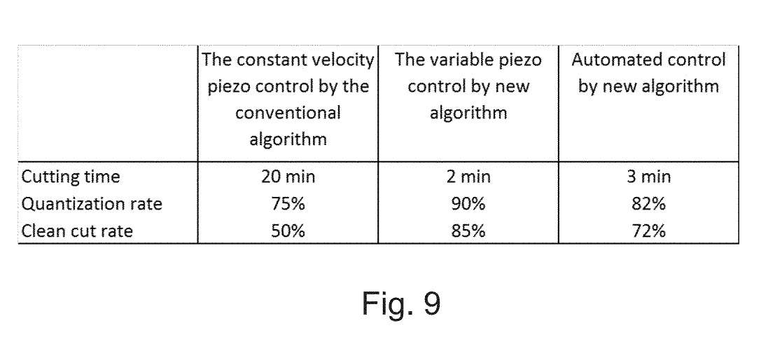

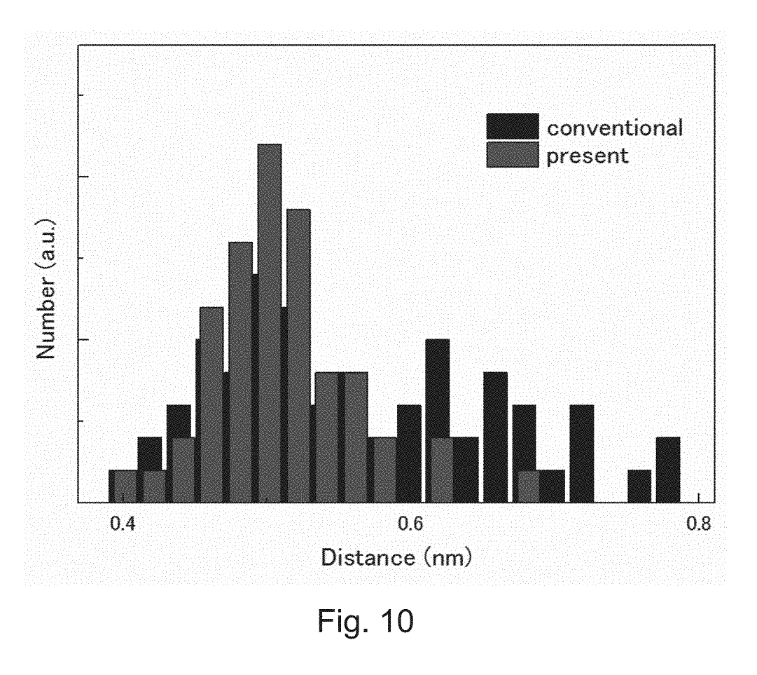

In some cases, improved nanoelectrode creation and calibration may be automated. FIG. 8A shows current and piezo voltage traces when using a conventional method. FIG. 8B shows current and piezo voltage traces generated using an automated system of the present disclosure. The results of the improved method are higher yield and more rapid breaking and setting of nanoelectrode pairs. FIG. 9 shows a table comparing the performance of nanoelectrodes generated by a manual method and nanoelectrodes generated by an automated method. FIG. 10 shows a gap distance distribution after breakage, using an automated protocol as compared with a manual protocol. As shown in the figure, the distribution is tighter when using an automated protocol, providing further evidence of a superior break. Methods, operations and protocols disclosed herein may be performed automatically (e.g., at least partially automated) or manually.

When measurement of the current (e.g., tunnel current) is continued, the sharpness of the shape of an electrode tip of a pair of nanoelectrodes may deteriorate with the passage of time. Thus, in some cases, it may be preferable to shape an electrode tip, by the repeated breakage and joining processing that is performed when forming the pair of nanoelectrodes, for example, right before start of measurement.

In some cases, a biomolecule analysis apparatus may have mechanisms for forming nanoelectrodes, and hardware for data collection. However, a biomolecule analysis apparatus that operates only in an analysis mode may be possible. For such a biomolecule analysis apparatus, a separate device may be provided, which has an electrode formation mode function, and which may include a loading part in which a cartridge may be loaded. The cartridge may include a plate to be measured on which a pair of nano-electrodes may be pre-formed.

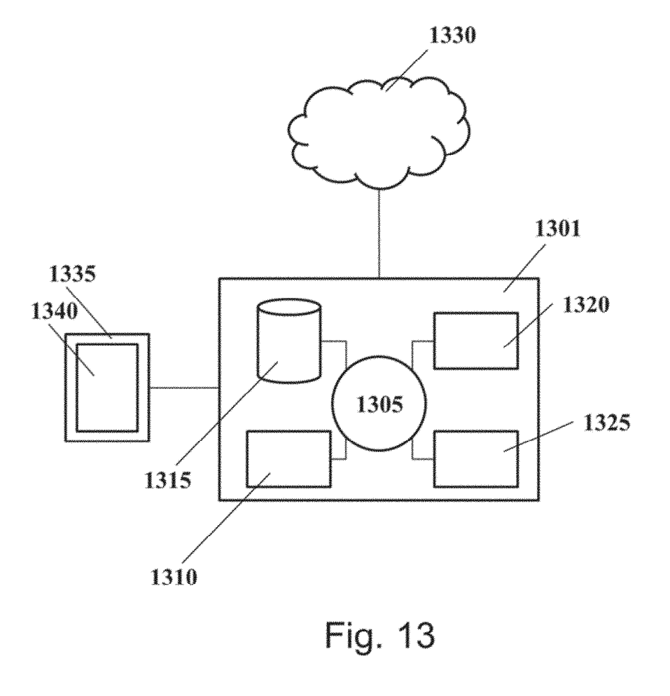

The present disclosure also provides computer control systems that are programmed or otherwise configured to implement methods provided herein, such as applying a voltage between a nanoelectrode pair or varying a gap distance between electrodes of a nanoelectrode pair. FIG. 13 shows a computer system 1301 that includes a central processing unit (CPU, also “processor” and “computer processor” herein) 1305, which can be a single core or multi core processor, or a plurality of processors for parallel processing. The computer system 1301 also includes memory or memory location 1310 (e.g., random-access memory, read-only memory, flash memory), electronic storage unit 1315 (e.g., hard disk), communication interface 1320 (e.g., network adapter) for communicating with one or more other systems, and peripheral devices 1325, such as cache, other memory, data storage and/or electronic display adapters. The memory 1310, storage unit 1315, interface 1320 and peripheral devices 1325 are in communication with the CPU 1305 through a communication bus (solid lines), such as a motherboard. The storage unit 1315 can be a data storage unit (or data repository) for storing data. The computer system 1301 can be operatively coupled to a computer network (“network”) 1330 with the aid of the communication interface 1320. The network 1330 can be the Internet, an internet and/or extranet, or an intranet and/or extranet that is in communication with the Internet. The network 1330 in some cases is a telecommunication and/or data network. The network 1330 can include one or more computer servers, which can enable distributed computing, such as cloud computing. The network 1330, in some cases with the aid of the computer system 1301, can implement a peer-to-peer network, which may enable devices coupled to the computer system 1301 to behave as a client or a server.

The CPU 1305 can execute a sequence of machine-readable instructions, which can be embodied in a program or software. The instructions may be stored in a memory location, such as the memory 1310. The instructions can be directed to the CPU 1305, which can subsequently program or otherwise configure the CPU 1305 to implement methods of the present disclosure. Examples of operations performed by the CPU 1305 can include fetch, decode, execute, and writeback.

The CPU 1305 can be part of a circuit, such as an integrated circuit. One or more other components of the system 1301 can be included in the circuit. In some cases, the circuit is an application specific integrated circuit (ASIC).

The storage unit 1315 can store files, such as drivers, libraries and saved programs. The storage unit 1315 can store user data, e.g., user preferences and user programs. The computer system 1301 in some cases can include one or more additional data storage units that are external to the computer system 1301, such as located on a remote server that is in communication with the computer system 1301 through an intranet or the Internet. The computer system 1301 can communicate with one or more remote computer systems through the network 1330.

Methods as described herein can be implemented by way of machine (e.g., computer processor) executable code stored on an electronic storage location of the computer system 1301, such as, for example, on the memory 1310 or electronic storage unit 1315. The machine executable or machine readable code can be provided in the form of software. During use, the code can be executed by the processor 1305. In some cases, the code can be retrieved from the storage unit 1315 and stored on the memory 1310 for ready access by the processor 1305. In some situations, the electronic storage unit 1315 can be precluded, and machine-executable instructions are stored on memory 1310.

The code can be pre-compiled and configured for use with a machine having a processor adapted to execute the code, or can be compiled during runtime. The code can be supplied in a programming language that can be selected to enable the code to execute in a pre-compiled or as-compiled fashion.

The computer system 1301 can be programmed or otherwise configured to regulate one or more parameters, such as the voltage applied across electrodes of a nanoelectrode pair, tensile stress applied on a region of a metal substrate, and time period for signal acquisition.

Aspects of the systems and methods provided herein, such as the computer system 1301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1301 can include or be in communication with an electronic display 1335 that comprises a user interface (UI) 1340 for providing, for example, signals from a chip with time. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1305.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of forming a nanogap electrode pair, comprising:
(a) providing a metal substrate having a region configured to form a gap;
(b) applying a voltage across said region of said metal substrate; and
(c) applying tensile stress on said region of said metal substrate to form said gap by controlling an expansion rate of said region along a direction of said tensile stress,
wherein said tensile stress is applied until a conductance of said metal substrate is less than 7 G₀.

2. The method of claim 1, wherein (c) is performed while measuring said conductance of said metal substrate.

3. The method of claim 1, wherein said metal substrate is a metal wire.

4. The method of claim 1, wherein (c) comprises:
(i) when said conductance is greater than or equal to 7 G₀, applying said tensile stress to provide a first expansion rate of said region;
(ii) when said conductance is less than 7 G₀, applying said tensile stress to provide a second expansion rate of said region, which second expansion rate is greater than said first expansion rate; and
(iii) when said conductance is less than or equal to 3 G₀, applying said tensile stress to provide a third expansion rate of said region, which third expansion rate is less than said second expansion rate.

5. The method of claim 4, wherein said tensile stress is applied to provide said third expansion rate when said conductance is between 1 G₀ and 3 G₀.

6. The method of claim 1, wherein said gap has a spacing from 0.5 nm to less than a molecular diameter of a biomolecule.

7. A method of setting a G₀ gap distance between a pair of nanoelectrodes of a mechanically controlled break junction, comprising:
(a) applying a voltage between said pair of nanoelectrodes;
(b) varying a gap distance between said pair of nanoelectrodes, which varying includes applying one or more cycles of alternating motion to increase and reduce said gap distance, and wherein individual electrodes of said pair of nanoelectrodes do not reconnect when said gap distance is reduced;
(c) measuring a current between said pair of nanoelectrodes; and
(d) calculating said gap distance as a function of a plurality of data sets of said gap distance and said current.

8. The method of claim 7, wherein said current includes tunneling current.

9. The method of claim 7, wherein said varying comprises a deceleration of a rate at which said gap distance is varied prior to a reversal in a direction in which said gap distance is varied.

10. A method for forming a nanoelectrode pair, comprising:
(a) forming a metal wire coated with an electrically insulating material; and
(b) forming said nanoelectrode pair from said metal wire, wherein said nanoelectrode pair has a gap with a spacing from about 0.5 nanometers (nm) to 10 nm.

11. The method of claim 10, wherein molecules of said electrically insulating material are bonded to multiple atoms of said metal wire.

12. The method of claim 10, wherein (b) comprises subjecting said metal wire to stress.

13. The method of claim 10, wherein said nanoelectrode pair has a gap that is configured to detect a current upon flow of a biomolecule through said gap.

14. A method for forming a gap spacing in a nanoelectrode pair, comprising:
(a) providing said nanoelectrode pair having tips;
(b) rejoining said tips of said nanoelectrode pair,
(c) rebreaking said nanoelectrode pair to provide said tips; and
(d) repeating (b) and (c) at least five times, thereby creating a reformed nanoelectrode pair having said gap spacing.

15. The method of claim 14, wherein (a) comprises providing a metal wire and breaking said metal wire, wherein said breaking and rejoining are performed using an actuator, and wherein an average velocity of said actuator during said breaking is less than an average velocity of said actuator during said rejoining.

16. A method for providing a reformed nanoelectrode pair, comprising:
(a) providing a nanoelectrode pair having a degraded performance, which degraded performance is characterized by increased background noise, wherein said nanoelectrode pair includes separate electrodes with tips;
(b) rejoining said tips of said nanoelectrode pair to form a rejoined unit;
(c) breaking said rejoined unit to reform said nanoelectrode pair; and
(d) repeating (b) and (c) at least once to provide said reformed nanoelectrode pair.

17. The method of claim 16, further comprising, prior to (b) measuring said degraded performance in said nanoelectrode pair.

18. The method of claim 16, wherein (d) comprises repeating (b) and (c) at least twice.

19. The method of claim 16, wherein (d) comprises repeating (b) and (c) at three times.

20. The method of claim 16, wherein said reformed nanoelectrode pair has a gap with a spacing from 0.5 nanometers (nm) to 2 times a molecular diameter of a biomolecule.
